(19) United States
(12) Patent Application Publication
(10) Pub. No.: US 2022/0240849 A1
(43) Pub. Date: Aug. 4, 2022

(54) WEARABLE DEVICES EQUIPPED WITH BLOOD ALCOHOL CONTENT SAMPLE ANALYZER, INDICATIVE LED SIGNALS, AND BLUETOOTH CAPABILITIES

(71) Applicants: Cade Wolfram Spector, Raleigh, NC (US); Jack Edward Mulcrone, Vernon Hills, IL (US); Cole Garrett Spector, Raleigh, NC (US); Devin Patrick Mulcrone, Vernon Hills, IL (US); Sawyer Beckett Spector, Raleigh, NC (US)

(72) Inventors: Cade Wolfram Spector, Raleigh, NC (US); Jack Edward Mulcrone, Vernon Hills, IL (US); Cole Garrett Spector, Raleigh, NC (US); Devin Patrick Mulcrone, Vernon Hills, IL (US); Sawyer Beckett Spector, Raleigh, NC (US)

(21) Appl. No.: 17/587,566

(22) Filed: Jan. 28, 2022

Related U.S. Application Data
(60) Provisional application No. 63/143,530, filed on Jan. 29, 2021.

(51) Int. Cl. A61B 5/00; A61B 5/08; A61B 5/097

(52) U.S. Cl. CPC A61B 5/4845; A61B 5/082; A61B 2560/04; A61B 5/681; A61B 5/742; A61B 5/097

(57) ABSTRACT

A wearable device for measuring intoxication of a user can include a band with a sample intake port, one or more processors or other circuitry that analyze a sample from the user, the sample being inducted into the sample intake port, and determine a level of intoxication of the user based on an analysis of the sample, and an indicator that visually displays the level of intoxication of the user on an exterior surface of the band.

### CROSS-REFERENCE TO RELATED APPLICATION

This application claims priority to U.S. Provisional Patent Application No. 63/143,530, filed Jan. 29, 2021, the disclosure of which is incorporated by reference herein in its entirety.

### TECHNICAL FIELD

The subject matter disclosed herein relates generally to electrospray emitter devices. More particularly, the subject matter disclosed herein relates to the use of such electrospray emitters as thrusters for moving and/or maneuvering objects to which such thrusters are attached, including in ambient environments where there is no, or only negligible, ambient barometric pressure (e.g., in a vacuum), such as in space.

### BACKGROUND

Blood-alcohol-content (BAC) or blood-alcohol-concentration is a measurement of the amount of alcohol in a person's blood stream. BAC is typically measured as the amount of alcohol in the person's blood as a percentage of the total blood in a sample. For example, if a person has a BAC of 0.10% that means their blood contains 1-part alcohol for every 1000 parts of blood. Although not a foolproof measure of a person's level of inebriation, a person's BAC is the standard measure for determining inebriation in various settings, such as criminal justice.

The typical non-invasive (i.e., does not require a blood draw for analysis) device used to measure a person's BAC is called a breathalyzer. Breathalyzers are bulky, inconvenient, and contain a stigma related to alcoholism and drunk driving. Although these constraints are not particularly cumbersome to law enforcement officers when performing routine traffic stops and drunk-driving checks, they can be a hindrance in the personal consumer marketplace (i.e., non-law enforcement consumers). Currently, there exists standard hand-held breathalyzers, portable breathalyzers, iPhone accessory breathalyzers, and unreliable skin absorption technology. However, these devices share some of the same constraints as the breathalyzers used by law enforcement.

Furthermore, there exists an ever-present danger with irresponsible alcohol consumption. College students, bar and dance club patrons, sporting event spectators and many other consumers of alcohol find it difficult or impossible to determine if they are inebriated to a point where they are legally unable to drive or otherwise determine their level of inebriation without resorting to using the cumbersome devices described above. Additionally, the stigma associated with using a bulky breathalyzer can be a deterrent to obtaining a measurement of one's BAC when it is needed. Thus, there is a need for an easy to transport, convenient, and fashionable device that will make it easier to measure one's BAC without the constraints discussed above.

### SUMMARY

In accordance with this disclosure, wearable devices and systems for measuring intoxication of a user are provided. In one aspect, a wearable device for measuring intoxication of a user is provided. In some embodiments, the wearable device comprises: a band comprising a sample intake port; a detachable induction tube, wherein the induction tube is configured to extend outward from the band when inserted into the sample intake port; and one or more processors or other circuitry configured to analyze a sample from the user, the sample being inducted into the sample intake port, and indicate a level of intoxication of the user based on an analysis of the sample.

In some embodiments, the band further comprises: two semi-circle halves that are pulled together with elastic bands and held together with magnets. In some embodiments, the band further comprises an integrated holster configured for securely holding the detachable induction tube. In some embodiments, the integrated holster is configured such that at least a portion of the detachable induction tube is hidden within the band. In some embodiments, the band is configured to be worn on the wrist, hand, or one or more finger of the user. In some embodiments, the band further comprises one or more light emitting diodes (LEDs). In some embodiments, the one or more LEDs are configured to indicate a level of intoxication of the user based on analysis of the sample provided by the user; and wherein the indication of the level of intoxication of the user is based on user input settings. In some embodiments, the one or more LEDs comprises four LEDs. In some embodiments, the four LEDs are each a different color to indicate various levels of intoxication. In some embodiments, an intoxication level represented by each of the four LEDs can be set by the user using a mobile application in communication with the wearable device via Bluetooth, Wi-Fi, or a mobile communications data network.

Moreover, in some embodiments, the wearable device is in communication with a mobile application via Bluetooth, Wi-Fi, or a mobile communications data network. In some embodiments, the mobile application is operated by a mobile phone, tablet, personal digital assistant, or laptop. In some embodiments, the mobile application comprises a signature feature such that sexual consent can be given via the signature feature if the user is not intoxicated. In some embodiments, the wearable device is configured to receive the sample from the user via the detachable induction tube and the sample intake port; wherein the sample is configured to be analyzed by the one or more processors or other circuitry; and wherein an analysis of the sample is configured to be transmitted to the mobile application for determining the level of intoxication of the user based on the analysis.

In some embodiments, the level of intoxication is based on the amount of alcohol, tetrahydrocannabinol (THC), Rohypnol, Benzodiazepine, Ketamine, or opioids in the sample.

In another aspect of the present disclosure, a system is provided comprising: a computing device operating a software application; and a wearable device in communication with the computing device and the software application, the wearable device being configured for measuring intoxication of a user and comprising: a band comprising a sample intake port; a detachable induction tube, wherein the induction tube is configured to extend outward from the band when inserted into the sample intake port; and one or more processors or other circuitry configured to analyze a sample from the user, the sample being inducted into the sample intake port, and indicate a level of intoxication of the user based on an analysis of the sample.

In some embodiments, the computing device is a mobile device, and the software application is a mobile software application. In some embodiments, the wearable device is configured to communicate with the computing device via Bluetooth, Wi-Fi, or a mobile data communication network. In some embodiments, the band further comprises: two semi-circle halves that are pulled together with elastic bands and held together with magnets. In some embodiments, the band further comprises an integrated holster configured for securely holding the detachable induction tube. In some embodiments, the integrated holster is configured such that at least a portion of the detachable induction tube is hidden within the band. In some embodiments, the band is configured to be worn on the wrist, hand, or one or more finger of the user.

In some embodiments, the band further comprises one or more light emitting diodes (LEDs). In some embodiments, the one or more LEDs are configured to indicate a level of intoxication of the user based on analysis of the sample provided by the user; and wherein the indication of the level of intoxication of the user is based on user input settings. In some embodiments, the one or more LEDs comprises four LEDs. In some embodiments, the four LEDs are each a different color to indicate various levels of intoxication. In some embodiments, an intoxication level represented by each of the four LEDs can be set by the user using a mobile application in communication with the wearable device via Bluetooth, Wi-Fi, or a mobile communications data network.

In some embodiments, the wearable device is in communication with a mobile application via Bluetooth, Wi-Fi, or a mobile communications data network. In some embodiments, the mobile application is operated by a mobile phone, tablet, personal digital assistant, or laptop. In some embodiments, the mobile application comprises a signature feature such that sexual consent can be given via the signature feature if the user is not intoxicated. In some embodiments, the wearable device is configured to receive the sample from the user via the detachable induction tube and the sample intake port; wherein the sample is configured to be analyzed by the one or more processors or other circuitry; and wherein an analysis of the sample is configured to be transmitted to the mobile application for determining the level of intoxication of the user based on the analysis. In some embodiments, the level of intoxication is based on the amount of alcohol, tetrahydrocannabinol (THC), Rohypnol, Benzodiazepine, Ketamine, or opioids in the sample.

According to another example embodiment, a wearable device for measuring intoxication of a user is provided herein. Such a wearable device comprises: a band comprising a sample intake port; one or more processors or other circuitry configured to analyze a sample from the user, the sample being inducted into the sample intake port, and to determine a level of intoxication of the user based on an analysis of the sample; and an indicator configured to visually display the level of intoxication of the user.

According to another example embodiment, a wearable device for measuring intoxication of a user is provided herein. Such a wearable device comprises: a band comprising a sample intake port; one or more processors or other circuitry configured to analyze a sample from the user, the sample being inducted into the sample intake port, and to determine a level of intoxication of the user based on an analysis of the sample; an indicator configured to visually display the level of intoxication of the user; and a detachable induction tube configured to extend outward from the band when inserted into the sample intake port.

According to another example embodiment, a wearable device for measuring intoxication of a user is provided herein. Such a wearable device comprises: a band comprising a sample intake port; one or more processors or other circuitry configured to analyze a sample from the user, the sample being inducted into the sample intake port, and to determine a level of intoxication of the user based on an analysis of the sample; an indicator configured to visually display the level of intoxication of the user; and a detachable induction tube configured to extend outward from the band when inserted into the sample intake port; wherein the band further comprises an integrated holster configured for securely holding the detachable induction tube in a position such at least a portion of the detachable induction tube is hidden within the band.

According to another example embodiment, a wearable device for measuring intoxication of a user is provided herein. Such a wearable device comprises: a band comprising a sample intake port; one or more processors or other circuitry configured to analyze a sample from the user, the sample being inducted into the sample intake port, and to determine a level of intoxication of the user based on an analysis of the sample; and an indicator configured to visually display the level of intoxication of the user; wherein the band comprises two semi-circular band portions that are interconnected at opposing ends thereof with a resilient member and held together with magnets.

According to another example embodiment, a wearable device for measuring intoxication of a user is provided herein. Such a wearable device comprises: a band comprising a sample intake port; one or more processors or other circuitry configured to analyze a sample from the user, the sample being inducted into the sample intake port, and to determine a level of intoxication of the user based on an analysis of the sample; and an indicator configured to visually display the level of intoxication of the user; wherein the band is configured to be worn on a wrist, hand, or one or more finger of the user.

According to another example embodiment, a wearable device for measuring intoxication of a user is provided herein. Such a wearable device comprises: a band comprising a sample intake port; one or more processors or other circuitry configured to analyze a sample from the user, the sample being inducted into the sample intake port, and to determine a level of intoxication of the user based on an analysis of the sample; and an indicator configured to visually display the level of intoxication of the user; wherein the indicator comprises one or more light emitting diodes (LEDs) on an exterior surface of the band.

According to another example embodiment, a wearable device for measuring intoxication of a user is provided herein. Such a wearable device comprises: a band comprising a sample intake port; one or more processors or other circuitry configured to analyze a sample from the user, the sample being inducted into the sample intake port, and to determine a level of intoxication of the user based on an analysis of the sample; and an indicator configured to visually display the level of intoxication of the user; wherein the indicator comprises one or more light emitting diodes (LEDs) on an exterior surface of the band; and wherein the indication of the level of intoxication of the user is based on user input settings.

According to another example embodiment, a wearable device for measuring intoxication of a user is provided herein. Such a wearable device comprises: a band comprising a sample intake port; one or more processors or other circuitry configured to analyze a sample from the user, the sample being inducted into the sample intake port, and to determine a level of intoxication of the user based on an analysis of the sample; and an indicator configured to visually display the level of intoxication of the user; wherein the indicator comprises one or more light emitting diodes (LEDs) on an exterior surface of the band; and wherein the one or more LEDs comprises a plurality of LEDs, each of which is a different color to indicate various levels of intoxication.

According to another example embodiment, a wearable device for measuring intoxication of a user is provided herein. Such a wearable device comprises: a band comprising a sample intake port; one or more processors or other circuitry configured to analyze a sample from the user, the sample being inducted into the sample intake port, and to determine a level of intoxication of the user based on an analysis of the sample; and an indicator configured to visually display the level of intoxication of the user; wherein the indicator comprises one or more light emitting diodes (LEDs) on an exterior surface of the band; wherein the one or more LEDs comprises a plurality of LEDs, each of which is a different color to indicate various levels of intoxication; wherein the level of intoxication represented by each of the plurality of LEDs can be set by the user using a software application in communication with the wearable device via Bluetooth, WiFi, and/or a mobile communications data network; and wherein the wearable device is configured to transmit results of analysis of the sample to the software application for determining the level of intoxication of the user based on the analysis.

According to another example embodiment, a wearable device for measuring intoxication of a user is provided herein. Such a wearable device comprises: a band comprising a sample intake port; one or more processors or other circuitry configured to analyze a sample from the user, the sample being inducted into the sample intake port, and to determine a level of intoxication of the user based on an analysis of the sample; and an indicator configured to visually display the level of intoxication of the user; wherein the indicator comprises one or more light emitting diodes (LEDs) on an exterior surface of the band; wherein the one or more LEDs comprises a plurality of LEDs, each of which is a different color to indicate various levels of intoxication; wherein the level of intoxication represented by each of the plurality of LEDs can be set by the user using a software application in communication with the wearable device via Bluetooth, WiFi, and/or a mobile communications data network; wherein the wearable device is configured to transmit results of analysis of the sample to the software application for determining the level of intoxication of the user based on the analysis; and wherein the mobile application is stored in a memory of, and executed by one or more processors of, a mobile phone, tablet, personal digital assistant, or laptop.

According to another example embodiment, a wearable device for measuring intoxication of a user is provided herein. Such a wearable device comprises: a band comprising a sample intake port; one or more processors or other circuitry configured to analyze a sample from the user, the sample being inducted into the sample intake port, and to determine a level of intoxication of the user based on an analysis of the sample; and an indicator configured to visually display the level of intoxication of the user; wherein the indicator comprises one or more light emitting diodes (LEDs) on an exterior surface of the band; wherein the one or more LEDs comprises a plurality of LEDs, each of which is a different color to indicate various levels of intoxication; wherein the level of intoxication represented by each of the plurality of LEDs can be set by the user using a software application in communication with the wearable device via Bluetooth, WiFi, and/or a mobile communications data network; wherein the wearable device is configured to transmit results of analysis of the sample to the software application for determining the level of intoxication of the user based on the analysis; and wherein the mobile application comprises a signature feature such that sexual consent can be indicated via the signature feature only when the level of intoxication of the user is below a predetermined threshold.

According to another example embodiment, a wearable device for measuring intoxication of a user is provided herein. Such a wearable device comprises: a band comprising a sample intake port; one or more processors or other circuitry configured to analyze a sample from the user, the sample being inducted into the sample intake port, and to determine a level of intoxication of the user based on an analysis of the sample; and an indicator configured to visually display the level of intoxication of the user; wherein the level of intoxication is determined based on presence and/or concentrations of alcohol, tetrahydrocannabinol (THC), Rohypnol, Benzodiazepine, Ketamine, and/or opioids in the sample.

According to another example embodiment, a wearable device for measuring intoxication of a user is provided herein. Such a wearable device comprises: a band comprising a sample intake port; one or more processors or other circuitry configured to analyze a sample from the user, the sample being inducted into the sample intake port, and to determine a level of intoxication of the user based on an analysis of the sample; and an indicator configured to visually display the level of intoxication of the user; wherein the indicator comprises a display screen.

According to another example embodiment, a wearable device for measuring intoxication of a user is provided herein. Such a wearable device comprises: a band comprising a sample intake port; one or more processors or other circuitry configured to analyze a sample from the user, the sample being inducted into the sample intake port, and to determine a level of intoxication of the user based on an analysis of the sample; and an indicator configured to visually display the level of intoxication of the user; wherein the indicator comprises a display screen; and wherein the display screen is a touchscreen.

According to another example embodiment, a wearable device for measuring intoxication of a user is provided herein. Such a wearable device comprises: a band comprising a sample intake port; one or more processors or other circuitry configured to analyze a sample from the user, the sample being inducted into the sample intake port, and to determine a level of intoxication of the user based on an analysis of the sample; and an indicator configured to visually display the level of intoxication of the user; wherein the band is configured for attachment to a smartwatch; and wherein the indicator is a display of the smartwatch.

According to another example embodiment, a wearable device for measuring intoxication of a user is provided herein. Such a wearable device comprises: a band comprising a sample intake port; one or more processors or other circuitry configured to analyze a sample from the user, the sample being inducted into the sample intake port, and to determine a level of intoxication of the user based on an analysis of the sample; and an indicator configured to visually display the level of intoxication of the user; wherein the band comprises first and second housings attached rigidly to an interior surface of the band, the first housing comprising a power source and the second housing comprising an analysis compartment in fluidic communication with the sample intake port for receiving the sample within the analysis compartment.

According to another example embodiment, a wearable device for measuring intoxication of a user is provided herein. Such a wearable device comprises: a band comprising a sample intake port; one or more processors or other circuitry configured to analyze a sample from the user, the sample being inducted into the sample intake port, and to determine a level of intoxication of the user based on an analysis of the sample; and an indicator configured to visually display the level of intoxication of the user; wherein the band comprises first and second housings attached rigidly to an interior surface of the band, the first housing comprising a power source and the second housing comprising an analysis compartment in fluidic communication with the sample intake port for receiving the sample within the analysis compartment; and wherein the first and second housings have a shape such that a distance between midpoints of a radially inward facing surface of each of the first and second housings less than a diameter of the band.

According to another example embodiment, a wearable device for measuring intoxication of a user is provided herein. Such a wearable device comprises: a band comprising a sample intake port; one or more processors or other circuitry configured to analyze a sample from the user, the sample being inducted into the sample intake port, and to determine a level of intoxication of the user based on an analysis of the sample; and an indicator configured to visually display the level of intoxication of the user; wherein the band comprises first and second housings attached rigidly to an interior surface of the band, the first housing comprising a power source and the second housing comprising an analysis compartment in fluidic communication with the sample intake port for receiving the sample within the analysis compartment; wherein the first and second housings have a shape such that a distance between midpoints of a radially inward facing surface of each of the first and second housings less than a diameter of the band; and wherein the shape of the first and second housings resist rotation of the wearable device about a wrist of the user when the wearable device is positioned on, about, and/or around the wrist of the user.

According to another example embodiment, a system for determining a level of intoxication of a user is provided herein. Such a system comprises: a wearable device comprising: a band comprising a sample intake port; one or more processors or other circuitry configured to analyze a sample from the user, the sample being inducted into the sample intake port, and to determine a level of intoxication of the user based on an analysis of the sample; and an indicator configured to visually display the level of intoxication of the user. Such a system also comprises a computing device operating a software application. According to this example embodiment of the system, the wearable device is in communication with the computing device and the software application and is configured for determining the level of intoxication of the user.

According to another example embodiment, a system for determining a level of intoxication of a user is provided herein. Such a system comprises: a wearable device comprising: a band comprising a sample intake port; one or more processors or other circuitry configured to analyze a sample from the user, the sample being inducted into the sample intake port, and to determine a level of intoxication of the user based on an analysis of the sample; and an indicator configured to visually display the level of intoxication of the user. Such a system also comprises a computing device operating a software application. According to this example embodiment of the system, the wearable device is in communication with the computing device and the software application and is configured for determining the level of intoxication of the user and the computing device is a mobile device and the software application is a mobile software application.

Although some of the aspects of the subject matter disclosed herein have been stated hereinabove, and which are achieved in whole or in part by the presently disclosed subject matter, other aspects will become evident as the description proceeds when taken in connection with the accompanying drawings as best described hereinbelow.

### BRIEF DESCRIPTION OF THE DRAWINGS

FIG. 1 is an isometric view of a plurality of wearable devices for measuring intoxication of a user (e.g., the person wearing the wearable device).

FIG. 2 is a top view of one of the wearable devices shown in FIG. 1, in an expanded configuration for passage over the wearer's hand when putting on or removing the wearable device from their wrist.

FIG. 3 is a side view of the wearable device in the expanded configuration shown in FIG. 2

FIG. 4 is an isometric view of the wearable device in the expanded configuration shown in FIG. 2.

FIG. 5 is an isometric view of one of the wearable devices shown in FIG. 1, in a measuring configuration for determining the presence and/or concentration of a substance in the blood of a user.

FIG. 6 is another isometric view of the wearable device in the measuring configuration shown in FIG. 5.

FIG. 7 is a back view of the wearable device shown in FIG. 1, in a wearing configuration.

FIGS. 8 and 9 are respective views of one of the wearable devices shown in FIG. 1 worn about the wrist of a human.

FIG. 10 shows a wireless connection between an example embodiment of the wearable device disclosed herein and a computing device.

FIG. 11 is a perspective view of another example embodiment of a wearable device for measuring intoxication of a user.

FIG. 12 is a perspective view of another example embodiment of a wearable device for measuring intoxication of a user.

FIG. 13 is a perspective front view of another example embodiment of a wearable device for measuring intoxication of a user.

FIG. 14 is a perspective rear view of the wearable device shown in FIG. 13.

FIG. 15 is a perspective side internal view of the wearable device shown in FIG. 13.

FIG. 16 is a perspective front view of the wearable device shown in FIG. 13, in a curved configuration, such as when worn around a human wrist.

FIG. 17 is a perspective rear view of the wearable device shown in FIG. 13 in the curved configuration shown in FIG. 16.

FIG. 18 is a perspective side internal view of the wearable device shown in FIG. 13, in a measuring configuration.

FIG. 19 is a perspective view of another example embodiment of a smart watch with a system for measuring intoxication of the user integrated therein.

FIG. 20 is a perspective view of an example embodiment of a band for a wearable electronic device, the band comprising a system for measuring intoxication of the user integrated therein.

### DETAILED DESCRIPTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “about,” when referring to a value or to an amount of a composition, mass, weight, temperature, time, volume, concentration, percentage, etc., is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions. As used herein, the term “substantially,” when referring to a value, an activity, or to an amount of a composition, mass, weight, temperature, time, volume, concentration, percentage, etc., is meant to encompass variations of in some embodiments ±40%, in some embodiments ±30%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions. As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole. As used herein, the phrase “consisting essentially of” limits the scope of a claim to the specified materials or steps, plus those that do not materially affect the basic and novel characteristic(s) of the claimed subject matter. With respect to the terms “comprising”, “consisting of”, and “consisting essentially of”, where one of these three terms is used herein, the presently disclosed and claimed subject matter can include the use of either of the other two terms.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

New wearable devices and systems for measuring intoxication of a user are discussed herein. Each of the example embodiments disclosed herein are directed towards a system a system comprising a wearable device, which can be used, either alone or in conjunction with other components of the system, to determine a degree of intoxication of the user. The terms “user” and “wearer” may be used interchangeably herein, however, it is noted that “user” is a generally broader term and would include, for example, a person who uses the system while the wearable device is worn by another person. In general, for reasons of privacy, safety, and hygiene, it is envisioned that the systems disclosed herein are only used by the person wearing the wearable device, but the systems disclosed herein are not necessarily limited to such instances. Via the wearable device, the system is configured to obtain a breath sample from the user; analyze at least a portion of the chemical composition of the breath sample; determine, based on the chemical composition analysis, a blood alcohol content (BAC) of the user; and produce personalized results (e.g., via a coordinated smartphone application) for the user to increase safety and responsibility of the user.

The smartphone application is supplied with information (e.g., directly from the user or from a database containing the information, such as during initialization, or registration, of the application) and the determined BAC to produce a qualitative degree of intoxication (e.g., based on a concentration of alcohol or any other intoxicant that can be detected within the breath of the user). Based on a user-specified desired limit of intoxication, a signal (e.g., audible, visual, haptic, etc.) is transmitted to the wearer regarding the degree of intoxication produced. The information received regarding the user can include, for example, weight, height, gender, food intake history (e.g., within a prescribed time period), biological and/or genetic factors, and/or hydration information (e.g., water or other liquid intake over a prescribed time period).

In some embodiments, the signal is a light emitted from a light source, such as light emitting diode (LED). In such an example embodiment, a different color of light is emitted to provide information to the wearer regarding his/her degree of intoxication. For example, based on the desired limit of intoxication input by the user, the BAC detected for the user, and the information regarding the user, a green light may be emitted to indicate continued yet responsible intake of alcohol or any other suitable intoxicant; a yellow light may be emitted to indicate that the degree of intoxication is nearing the user-specified limit of intoxication, such that the user can reduce ingestion (e.g., drinking) of alcohol; a blue light may be emitted to indicate that the degree of intoxication has reached the user-specified limit of intoxication, such that the user can stop ingestion of alcohol; a red light may be emitted to suggest that the amount of alcohol consumed by the user has resulted in the user exceeding the user-specified limit of intoxication, such that the user can, for example, increase hydration (e.g., drink water) and seek medical help, as/if necessary.

In some embodiments, the systems disclosed herein are capable of detecting intoxicants capable of inducing an altered mental state, including, but not limited to, alcohol, tetrahydrocannabinol (THC), Rohypnol, Benzodiazepine, Ketamine, and/or opioids. In some embodiments, the systems disclosed herein may be configured to include a safe sex feature, in which, for example, consent for engaging in sexual acts is denied when the wearer has a BAC above a selectable level; such safe sex features of the systems disclosed herein can include, for example, signature consent form and photo options.

The accompanying figures of this application illustrate various aspects of example embodiments of the presently disclosed subject matter. Although the figures detail some representative features of the devices and systems of the presently disclosed subject matter, the figures and/or descriptions of such example embodiments do not limit the scope of the subject matter of the present disclosure to those features illustrated in the figures, but are merely illustrative.

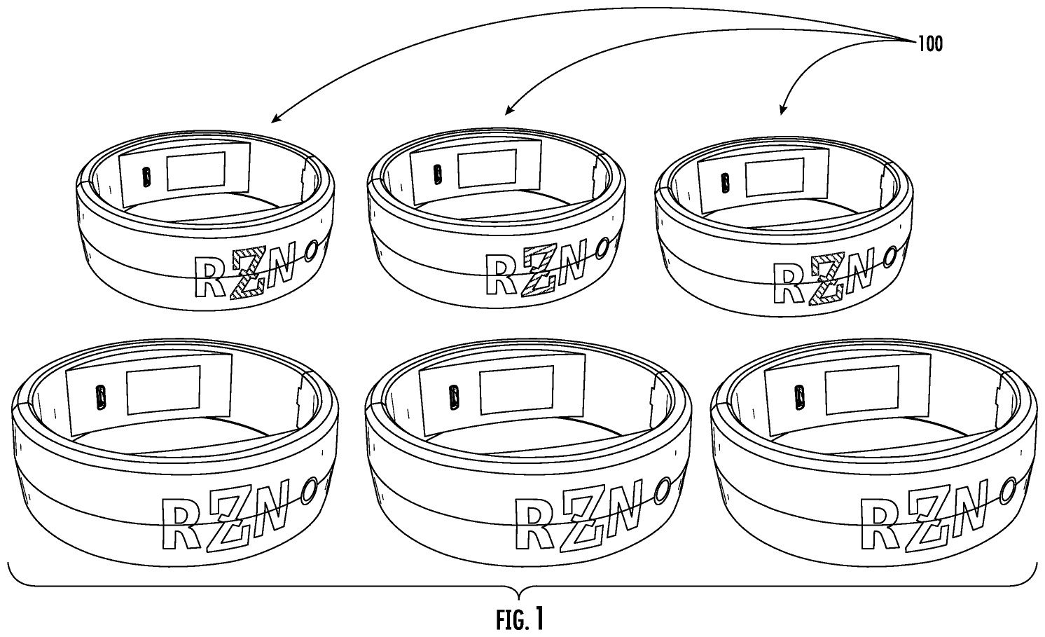

Referring to FIG. 1, illustrated therein are a plurality of variously sized example embodiments of wearable devices, generally designated 100, for measuring intoxication of a user/wearer. As shown, the wearable devices 100 comprise an outer band that is generally annularly-shaped (e.g., are in the shape of a band), having a general shape that is similar to a watch band or fitness tracker band. The wearable devices 100 have an outer contour, or shape, that is generally in the shape of a hollow cylinder, in which the length of the cylinder is less than (e.g., less than half of) the outer diameter of the wearable device. The wearable devices 100 have two housings, which are formed, respectively, on opposing surfaces of the inner radius of the outer band each other. A first housing is a power/communication housing. A second housing is an analytical housing. The housings have a generally flat surface (e.g., allowing for a curvature that approximates the upper and lower portions of the human wrist). Thus, when such a wearable device 100 is worn about and/or around the wrist of a human, the housings are shaped so as to prevent rotation of the wearable device 100 around the wrist of the wearer and to maintain a desired orientation (e.g., angular position) on the wrist of the wearer (e.g., so that the screen, or other suitable indicator, is oriented on the upper surface of the wrist).

As used herein, the “upper surface” of the wrist is the surface that, when a person's forearm and palm are positioned against a flat surface, such as a table, is not against (e.g., is pointed and/or further away from) the flat surface. Similarly, the “lower surface” of the wrist is the surface that, when a person's forearm and palm are positioned against a flat surface, such as a table, is against (e.g., is pointed towards, directly in contact with, and/or closest to) the flat surface. The housings and/or the outer band itself can be manufactured in a plurality of sizes, such that a wearable device 100 can be produced for any conceivable wrist size. The housings are shaped such that the distance between midpoints on the outer surfaces thereof is smaller than the inner diameter of the outer band.

The wearable device 100 can be made from, for example and without limitation, rubber, silicone, plastic, metal, cloth, polymers, combinations thereof, or from any other suitable product or material. In some embodiments, the outer band is formed from an elastic material and is configured to stretch over a human hand for positioning around a human wrist, or any other suitable structure (e.g., including anatomical and non-anatomical structures), such that the outer band has an undeformed (e.g., unstretched) inner diameter that is substantially the same as, or slightly bigger than (e.g., within 5%, within 10%, within 15%, within 20%, or within 25%) the user's wrist, while still being smaller than the greatest cross-sectional dimension (e.g., width) of the wearer's hand, such that the wearable device 100 will not slide over the wearer's hand without deformation of the outer band.

Also illustrated on several of the wearable devices 100 in FIG. 1 are light sources (e.g., light emitting diodes, or LEDs) that are on, or embedded within (e.g., at least partially or entirely, such that light from the light sources can be emitted through only one surface that is, for example, coplanar with the outer surface of the outer band) the outer band. The light sources are, in the example embodiment shown, in the shape of a letter “Z” and are configured for illumination at different colors (e.g., wavelengths) that correspond to and/or indicate to the user the level of intoxication measured by the wearable device 100.

In some embodiments, the wearable device 100 comprises one or more (e.g., a plurality of) light sources. The wearable devices 100 are advantageously configured to detect and also indicate, such as via illumination of the light sources, a level, or degree, of intoxication of the user, as determined by interaction (e.g., active or passive) of the wearer with the wearable device 100. The wearable devices 100 disclosed herein can use any quantity and/or any color of light source, without limitation, and the level of intoxication of the user can be a user-selectable and/or user-adjustable value. The wearable devices 100 disclosed herein are further advantageously capable of establishing a wireless connection (e.g., via any suitable wireless communication protocol, including, for example, WiFi®, Bluetooth®, radio frequency ID (RFID), near-field communication (NFC), and the like) with a mobile computing device (e.g., smartphone or other suitable electronic computing device) on which is stored and executed a software application configured to receive and transmit data with the wearable device 100.

In some embodiments, the mobile software application is configured such that the user can select one or more different levels of intoxication to be associated with the illumination of each light source, or combinations of light sources. For example and without limitation, the user can completely customize the light source illumination setting such that illumination of each light source indicates a different level of intoxication, all or some the levels of intoxication being user-selected and/or predefined. For example and without limitation, the mobile software application can be configured such that each light source on the wearable device 100 has an adjustable or defined setting, which is correlated to blood alcohol content (BAC) or any other suitable and/or desired level of intoxication, assigned to it and the user can determine, in the mobile software application illumination settings, to illuminate certain light sources, or combinations thereof, at a certain level of intoxication. By way of non-limiting illustrative example, it is contemplated that a user may set, in the mobile software application, illumination of a green light source when a level of intoxication correlated to a BAC value of less than or equal to 0.07 is detected by the wearable device 100; illumination of a yellow light source when a level of intoxication correlated to a BAC of greater than 0.08 (e.g., the threshold BAC value for intoxication in the United States when operating a vehicle) is detected by the wearable device 100; and illumination of a red light source when a level of intoxication correlated to a BAC value of 0.12 is detected by the wearable device 100. In this illustrative example, the illumination of the red light source when the level of intoxication correlated to a BAC value of 0.12 or greater may be used to indicate that the user should cease ingestion of the intoxicant and, if necessary, seek help. In some example embodiments, the wearable device 100 comprises further other light sources, which may be illuminated based on, for example, user settings or based on detection by the wearable device 100 of one or more predefined conditions.

In some embodiments, the wearable device 100 is advantageously capable of detecting multiple types of intoxication (e.g., intoxication from different sources) at the same time (e.g., due to simultaneous intoxication). For example and without limitation, the wearable device 100 can be used to determine a level of alcohol and tetrahydrocannabinol (THC) in the blood. In some embodiments of the wearable device 100, illumination of a green light source may be initiated when THC is detected in the sample (e.g., exhaled breath) obtained at the wearable device 100 from the wearer, while illumination of light sources emitting other colors may be initiated based on detection by the wearable device 100 of any of a plurality of levels of intoxication (e.g., correlated to a BAC value) due to ingestion of alcohol.

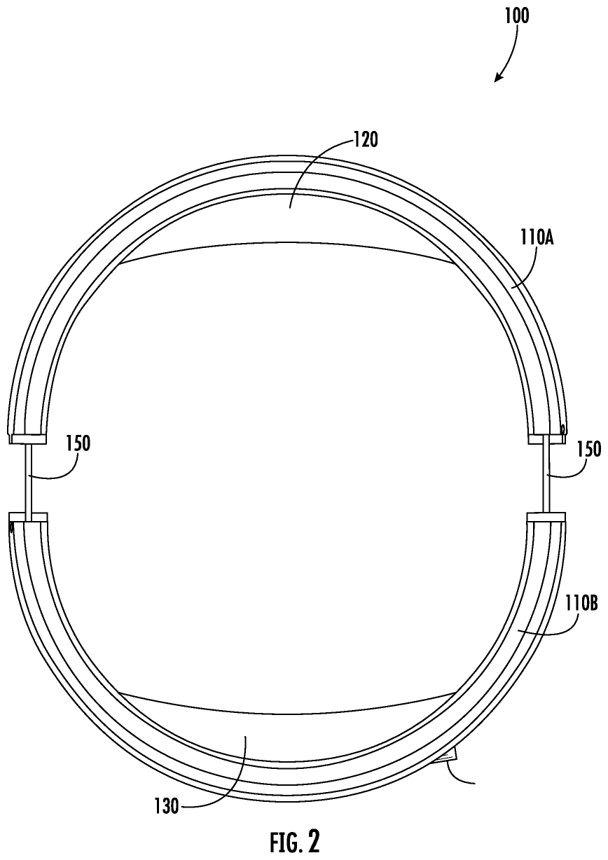

FIGS. 2 through 7 show various aspects of an example embodiment of one of the wearable devices, generally designated 100, shown in FIG. 1. In the example embodiment shown, the wearable device, generally designated 100, has a generally annular shape and comprises an outer band formed from two (e.g., first and second) band portions 110A, 110B, which are each generally in the shape of a semicircle. The two band portions 110A, 110B can comprise, for example and without limitation, any of plastic, rubber, silicone, and/or metal. Any suitable material may be used in the construction of any portion of the two band portions 110A, 110B.

The band portions 110A, 110B can be connected together at the opposing ends thereof by a resilient member 150. The resilient member 150 is advantageously formed from a material that can readily and repeatedly undergo elastic deformation over a defined range of movement of the first band portion 110A away from the second band portion 110B when the wearable device passes over the hand of the wearer to be worn on the wrist of such wearer. Materials that would undergo plastic deformation over such range of movement should not be used for the resilient member 150. Thus, the resilient member 150 advantageously comprises an elastic, or elastomeric, material that will return to an original length (e.g., the length of the resilient member 150 before it was ever stretched) when the resilient member 150 is stretched by no more than the specified range or movement of the first and second band portions 110A, 110B. Since the resilient members 150 are each connected between opposing ends of the first and second band portions 110A, 1108, the resilient material interconnects the first and second band portions 110A, 110B together and prevents them from being fully detached from one another when, for example, the wearable device 100 is in the opened position (see, e.g., FIGS. 2-4) passing over the hand of the wearer to put on or take off the wearable device 100.

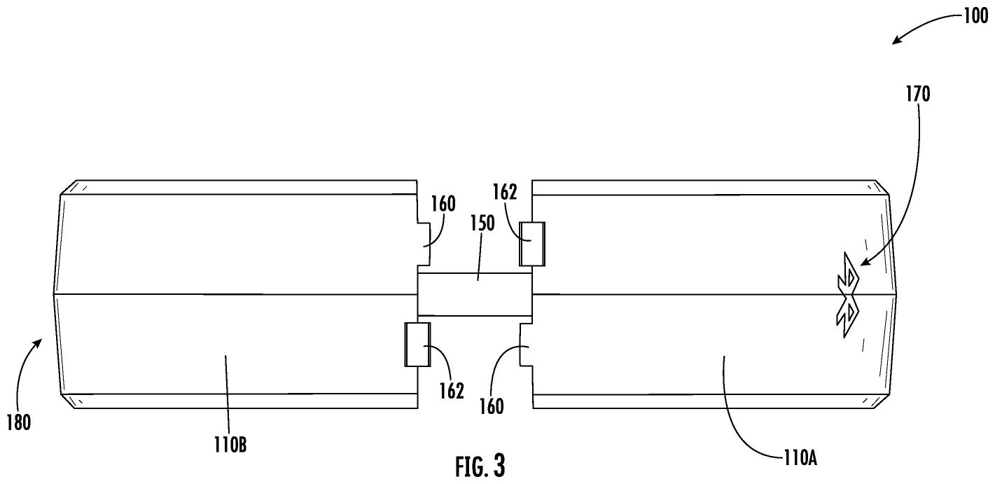

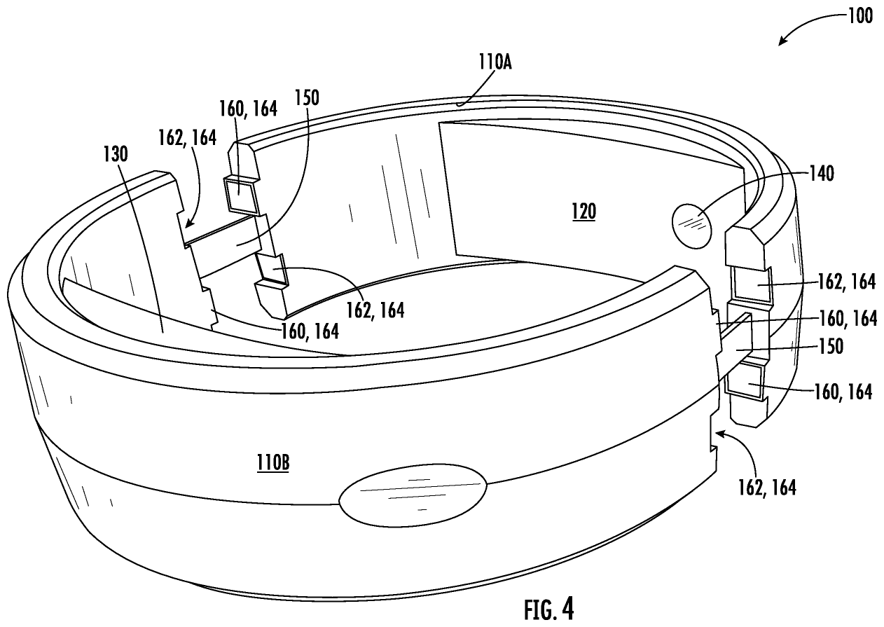

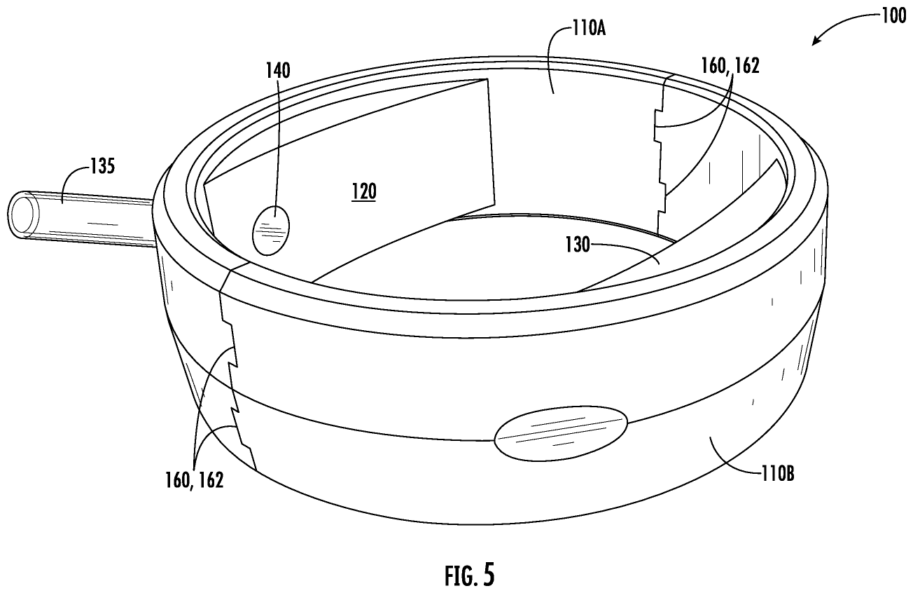

As shown in FIGS. 3 and 4, the first and second band portions 110A, 110B are shaped so as to interlock with each other in a closed position (see, e.g., FIGS. 5-7). Thus, at each end of both of the first and second band portions 110A, 110B there are formed a recess 160 and a protrusion 162. The orientation of the recess 160 and the protrusion 162 at each of the ends of the first band portion 110A is opposite to the orientation of the recess 160 and the protrusion 162 are each of the ends of the second band portion 110B. As such, a recess 160 in a first end of the first band portion 110A is positioned substantially coaxial with a corresponding protrusion 162 in a first end of the second band portion 110B and a protrusion 162 in the first end of the first band portion 110A is positioned substantially coaxial with a corresponding recess 160 in the first end of the second band portion 110B. Similarly, a recess 160 in a second end of the first band portion 110A is positioned substantially coaxial with a corresponding protrusion 162 in a second end of the second band portion 110B and a protrusion 162 in the second end of the first band portion 110A is positioned substantially coaxial with a corresponding recess 160 in the second end of the second band portion 110B. In the preceding description of the respective first and second ends of the interconnection of the first and second band portions 110A, 110B, the first ends of both of the first and second band portions 110A, 110B are connected to a same (e.g., a first) resilient member 150 and the second ends of both of the first and second band portions 110A, 110B are connected to a same (e.g., a second) resilient member 150. In one or both of each of the recesses 160 and protrusions 162, there are provided magnets 164 that generate a magnetic force therebetween when a protrusion 162 is positioned within a corresponding recess 160, such that the magnetic force operates to clasp and connect together the first and second band portions 110A, 110B, such that the wearable device 100 has a generally circular, or annular, cross-sectional outer profile, or shape.

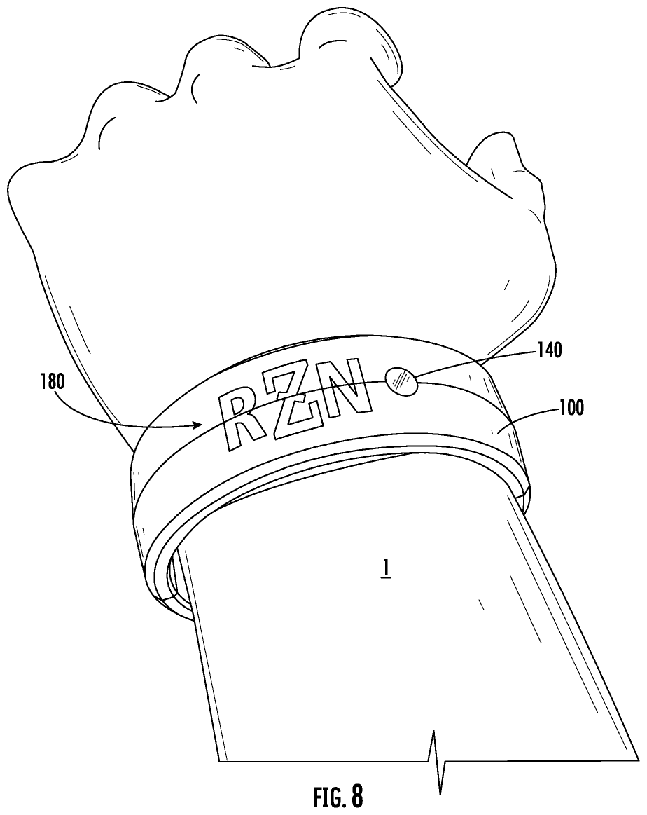

While it is shown in the accompanying example embodiments that the wearable device 100 is configured to be securely worn on, about, and/or around a user's wrist (see, e.g., FIGS. 8-10), the scope of the subject matter disclosed herein should not be construed as being limited to such an implementation. in some embodiments, the wearable device 100 may be sized, shaped, and designed for wearing on any suitable bodily appendage, such as, for example, the wrist, hand, or one or more fingers (i.e., the wearable device could be in the form of a ring) of the user. In some embodiments, the wearable device 100 can be configured as a necklace, so as to be worn around the neck of the user.

The wearable device 100 has, attached to the first band portion 110A, a first housing 120 and, attached to the second band portion 110B, a second housing 130. The first and second housings 120, 130 are formed, respectively, on opposing surfaces of the inner radius of the outer band, which is defined by assembly of the first and second band portions 110A, 110B together, from each other. The first and second housings 120, 130 may be attached to the respective first and second band portions 110A, 110B in any suitable manner. In some embodiments, the first and second housings 120, 130 are attached to the respective first and second band portions 110A, 1108 in a watertight manner. In some embodiments, first housing 120 and the first band portion 110A (e.g., a portion thereof) are formed as a unitary, or monolithic, structure and/or the second housing 130 and the second band portion 110B (e.g., a portion thereof) are formed as a unitary, or monolithic structure. The first housing 120 is an analytical housing, in which the contents of the breath of the user are analyzed to determine, at least in part, a level of intoxication of the user. The second housing 130 is a power/communication housing, in which is provided a battery 132 (e.g., a removable or sealed battery) and a power/data interface port 134, via which information can be exchanged in a wired manner with the wearable device 100 and the battery 132 can be recharged. The first and second housings 120, 130 have a generally flat surface (e.g., allowing for a curvature that approximates the upper and lower portions of the human wrist). Thus, when such a wearable device 100 is worn about and/or around the wrist of a human, as shown in FIGS. 8-10, the first and second housings 120, 130 are shaped so as to prevent rotation of the wearable device 100 around the wrist of the wearer and to maintain a desired orientation (e.g., angular position) of the wearable device 100 on the wrist of the wearer (e.g., so that a screen, or other suitable indicator, is oriented on the upper surface of the wrist).

An indicator can also be away from the wearable device such as with a wireless connection to an external device configured to receive a signal from the wearable device and to provide a display showing the signal. As an example for when the indicator is part of the wearable device, an indicator 180, which can be in the form of a screen, one or more light sources (e.g., light emitting diodes, or LEDs) or any other suitable indicator type by which information can be transmitted visually to the user, is provided on the first band portion 110A. In the example embodiment, the indicator 180 is in a form of one or more (e.g., a plurality of) LEDs that are on, or embedded within (e.g., at least partially or entirely, such that light from the light sources can be emitted through only one surface that is, for example, coplanar with the outer surface of the outer band) the outer surface of the first band portion 110A. In the example embodiment shown, the LED(s) of the indicator 180 are in the shape of a letter “Z” and are configured for illumination to emit different colors (e.g., wavelengths) that correspond to and/or indicate to the user the level of intoxication of the user, as measured by the wearable device 100.

The wearable devices 100 comprise a controller (e.g., a processor in communication with a memory device, on which are stored executable instructions for determining a level of intoxication of the user) advantageously configured to detect and also indicate, such as via illumination of a portion of the indicator 180, a level, or degree, of intoxication of the user, as determined by interaction (e.g., active or passive) of the wearer with the wearable device 100. The wearable devices 100 disclosed herein can use any quantity and/or any color of light source, without limitation, and the level of intoxication of the user can be a user-selectable and/or user-adjustable value. The wearable device 100 also includes a wireless transceiver 170, by which it is capable of establishing a wireless connection (e.g., via any suitable wireless communication protocol, including, for example, WiFi®, Bluetooth®, radio frequency ID (RFID), near-field communication (NFC), and the like) with a mobile computing device (e.g., smartphone or other suitable electronic computing device) on which is stored and executed a software application configured to receive and transmit data with the wearable device 100.

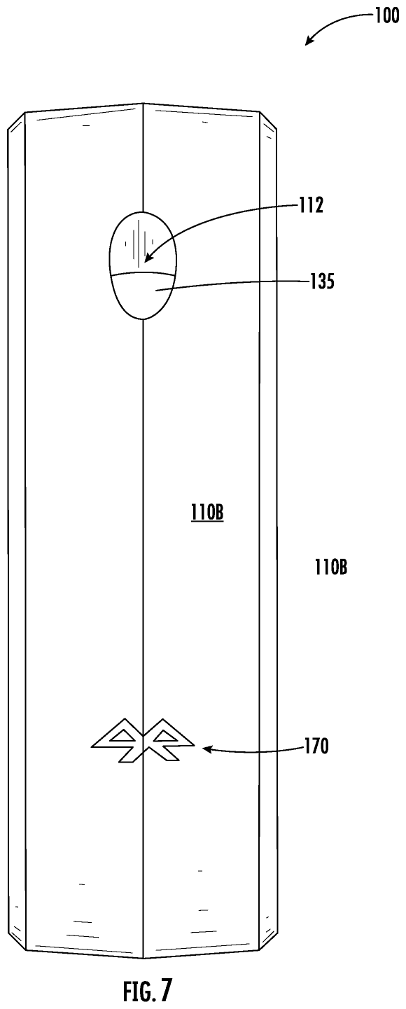

As shown in FIG. 7, the wearable device 100 comprises a holster, generally designated 112, formed integrally (e.g., in a unitary, or monolithic, manner) in the second band portion 110B. The holster 112 is configured for securely holding a detachable induction, or intake, tube 135. As shown, the holster 112 is configured such that at least a portion of the detachable induction tube 135 is visually occluded (e.g., hidden) within the second band portion 110B when the induction tube 135 is inserted within the holster 112. The holster 112 is a recess that has substantially similar dimensions to the outer dimensions of the induction tube 135 (e.g., such that the inner walls of the holster 112 are expanded upon insertion of the induction tube 135 therein, in the manner of a frictional, or interference, fit). The induction tube 135 is shaped such that it can slide in and out (e.g., upon manipulation by a user) of the integrated holster 112. In some embodiments, the induction tube 135 and the holster have a substantially similar curvature along the lengths thereof. In some embodiments, the integrated holster 112 comprises a lid or cap or other device to enclose the end of the induction tube 135 that protrudes to some degree from the open end of the holster 112, so as to prevent dirt and other debris from entering into the integrated holster, thereby keeping the induction tube clean.

In order to receive a breath sample from a user, the wearable device 100 comprises a sample intake port 140, which is formed in and through, at least partially, the first band portion 110A in the radial direction of the wearable device 100. The sample intake port 140 of the wearable device 100 is configured to receive an end of the detachable induction tube 135 therein. The user then exhales (e.g., by blowing) into the detachable induction tube 135 and the sample (i.e., the user's breath) flows through the detachable induction tube 135, into the sample intake port 140, and thereby into an analysis compartment contained within the first housing 120 of the wearable device 100. In some embodiments, the sample intake port 140 does not extend through the entire thickness of the first band portion 110, but instead has a terminal end within the first band portion 110 that prevents insertion of the induction tube 135 through the entire thickness of the first band portion 110A.

The distal, or interior (e.g., terminal), end of the sample intake port 140 is in fluidic (e.g., air) communication with the analysis compartment of the first band portion 110A, such that air flowing out of the distal end of the sample intake port 140 flows into the analysis compartment, in which chemical analysis of the sample is performed. Stated somewhat differently, after the sample is introduced into the sample intake port 140 via the induction tube 135, the sample intake port 140 comprises a conduit, hole, channel, etc. that is contained within the first band portion 110A and/or the first housing 120, which allows the sample to flow into the analysis compartment. In some embodiments, the side (e.g., circumferential) walls of the sample intake port 140 comprise one or more sensors and other circuitry, by which the wearable device is configured to detect and analyze the chemical composition of the sample. In some embodiments, the analysis compartment of the wearable device 100 comprises one or more processors or other circuitry (e.g., switches, sensors, logic gates, integrated circuits, Bluetooth® circuitry, Wi-Fi® circuitry, wireless data communications, and other circuitry) configured to analyze a sample from the user that is received through the sample intake port 140.

In some embodiments, the wearable device 100 can be operable without the use of the induction tube 135. In such an example embodiment, the user can exhale directly into the sample intake port 140; this can be especially useful in instances in which the induction tube 135 is dirty, lost, etc. In some embodiments, the induction tube 135 and, accordingly, the holster 112, can be omitted from the wearable device 100.

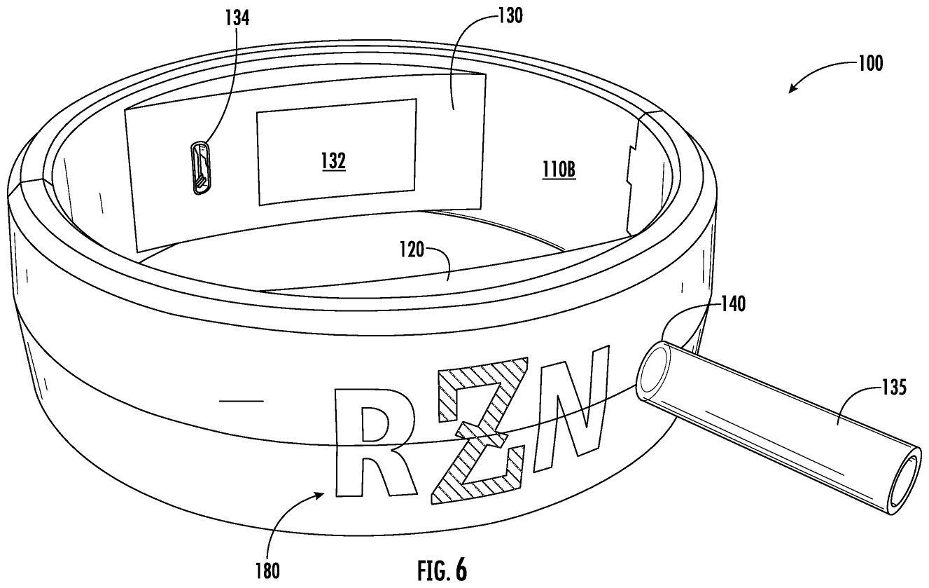

As shown in FIG. 6, the wearable device 100 comprises a rechargeable battery 132 to provide power (e.g., electric current at a specified voltage) to all of the electrical components of the wearable device. In embodiments having a rechargeable battery 132, a power/data interface port 134 is provided in the second housing 130, in electrical communication with the rechargeable battery 132. In the example embodiment shown in FIG. 6, the power/data interface port 134 is a micro-USB port, however, any suitable power/data connector type may be used for the power/data interface port 134 without limitation. The power/data interface port 134 is also, in some embodiments, advantageously connected to the processor(s), memory, and/or other circuitry in the first housing 120. In an example embodiment, this data connection between the power/data interface port 134 and the first housing 120 can be provided by electrical contacts (e.g., at one or more recess 160/protrusion 162 pairs) between the first and second band portions 110A, 110B.

In an example embodiment, the one or more processors and/or other circuitry within the first housing 120 is configured to analyze the sample received (e.g., into the sample intake port 140) from the user by interpreting sensor readings, which can be in the analysis compartment of the first housing 120 and/or about the perimeter of the sample intake port 140, when the sensors are exposed to the sample. As the sensors are exposed to the sample, the sensors detect the chemical composition of the sample and the one or more processors and/or other circuitry are configured to determine an amount (e.g., by volume, by weight, as a percentage, etc.) of each intoxicant detected in the sample.

After the one or more processors and/or other circuitry has analyzed the sample and determined the chemical composition of the sample, including an amount of intoxicant (if present) in the sample, the wearable device 100 is configured to communicate the results of such analysis of the sample to the user. As noted elsewhere herein, the results of the analysis can be displayed on the indicator 180. In some embodiments, the wearable device 100 is also configured to communicate with (e.g., to transmit the results of the analysis to) one or more computing devices, such as a mobile phone, laptop, personal digital assistant, tablet, server, etc. This communication between the wearable device 100 and the computing device(s) can be performed using any suitable wireless communication protocol, including, for example, Bluetooth®, WiFi®, or other wireless data communications network (i.e., 3G, LTE, 4G, 5G, etc.). After the results of the analysis of the sample is transmitted to the computing device(s), the computing device(s) can inform (e.g., via a graphical user interface (GUI) of software executed on such computing device) the user the level of intoxication that was detected as a result of the analysis, based on user input settings. For example and without limitation, the computing device can be a mobile phone operating a mobile software application in communication with the wearable device via Bluetooth®. In such an embodiment, when the mobile phone has received the results of the analysis of the sample, the mobile phone can, for example, indicate on the mobile software application the level of intoxication of the user that was detected and/or the mobile software application can, for example, instruct the wearable device 100 to indicate, via the indicator 180, the level of intoxication of the user as detected by the wearable device 100.

In some embodiments, the mobile software application is configured to receive data from the user to aid in a determination by the mobile software application as to the level of intoxication of the user (e.g., to use such user-input information to increase or decrease the level of intoxication that would be based solely on the chemical composition of the sample). For example, such user-input information can include weight, height, gender, recent food intake, biological/genetic risk, and/or hydration information. Using this user-input information, the mobile software application can use apply a weighting factor, using the user-input data, to the results of the analysis of the sample to determine the level of intoxication of the user.

For example and without limitation, in some embodiments, a mobile smartphone in communication with the wearable device 100 can receive analysis of the sample from the one or more processors of the wearable device 100 and combine that analysis with the user-input data to determine that the user is intoxicated at level equivalent to a “typical” or average person having a BAC value of 0.05 (e.g., the BAC value obtained from the measured data during sample analysis may be greater than or less than this BAC value).

User settings can be input into the mobile software application such that different actual/equivalent BAC levels (or THC levels, Rohypnol levels, levels of other chemical substances/drugs, etc.) are indicated by illumination of the indicator 180 of the wearable device 100 with a different color, or combination of colors.

For example, the user can input a setting in the mobile smartphone application such that a green LED of the indicator 180 will illuminate when the results of the analysis of the sample indicate a BAC value of less than 0.08. Thus, detection of an actual BAC value of 0.05 could be used to cause the mobile smartphone to instruct the wearable device to illuminate the green LED of the indicator 180. The one or more processors of the wearable device 100 receive (e.g., via wireless transceiver 170) this instruction from the mobile smartphone application and cause a corresponding illumination of, for example, one or more LEDs of the indicator 180. In some embodiments, the wearable device 100 is operable (e.g., to perform chemical analysis of a sample) without the use of a smartphone or any other electronic device in communication with the wearable device 100. In some embodiments, the wearable device 100 receives the user settings and/or user-input data and saves such information in the memory of the wearable device 100, such that the wearable device 100 can be operable without requiring communication with a mobile device or other electronic device during analysis of the sample and illumination of a corresponding LED (or LEDs, or a screen, display, etc.) of the indicator 180.

Continuing with the preceding example, the user can input a setting in the mobile smartphone application such that a yellow LED of the indicator 180 will illuminate when a BAC value of greater than 0.08 (e.g., between 0.08 and 0.1) is detected and a red LED of the indicator 180 will illuminate when a BAC value of greater than 0.1 is detected. These threshold values and illumination of specific colors corresponding to such threshold values are merely examples; it is advantageous for the user to be allowed to adjust and set the levels of intoxication for which the indicator 180 will be triggered to display information to the user regarding the user's level of intoxication. In some embodiments, the indicator 180 comprises one or more LEDs. For example and without limitation, the indicator 180 comprises four LEDs. In some embodiments, the indicator 180 comprises more than four LEDs. In some embodiments, the type, manner, and/or level of intoxication that is indicated by the illumination of each LED is fully customizable in the software application by the user. In some embodiments, the indicator 180 is a display screen (e.g., LCD, OLED, micro-LED, etc.).

Additionally, in some embodiments, the mobile software application can give a quantitative intoxication indication to the user. In some embodiments, the mobile software application can generate an informational message to display to the user (e.g., on the display screen of the mobile phone and/or, if so equipped, of the wearable device 100). An example of such an informational message could be as follows: “Sample indicates a BAC level of 0.08.”

As shown in FIGS. 5 and 6, in the example embodiment illustrated therein, when the detachable induction tube 135 is inserted into the sample intake port 140, the detachable induction tube 135 extends outward (e.g., radially) from the wearable device 100. This radially-extended orientation is advantageous at least for ergonomic reasons, to allow the user to freely blow into the detachable induction tube 135 while providing the sample for analysis. Once the user is finished blowing into the detachable induction tube 135, the user can dispose of the induction tube 135 or wash and insert the induction tube 135 into the holster 112.

FIG. 7 shows the wearable device 100 having a wireless transceiver 170 in the form of a Bluetooth® radio and receiver module. As discussed elsewhere herein, the wireless transceiver 170 can use any suitable wireless communication protocol, including Bluetooth®, WiFi®, wireless data communication connection, and/or any other suitable wireless communication protocol configured for enabling communication between the wearable device 100 and a computing device (i.e., a smartphone) and the software application stored and executed thereon.

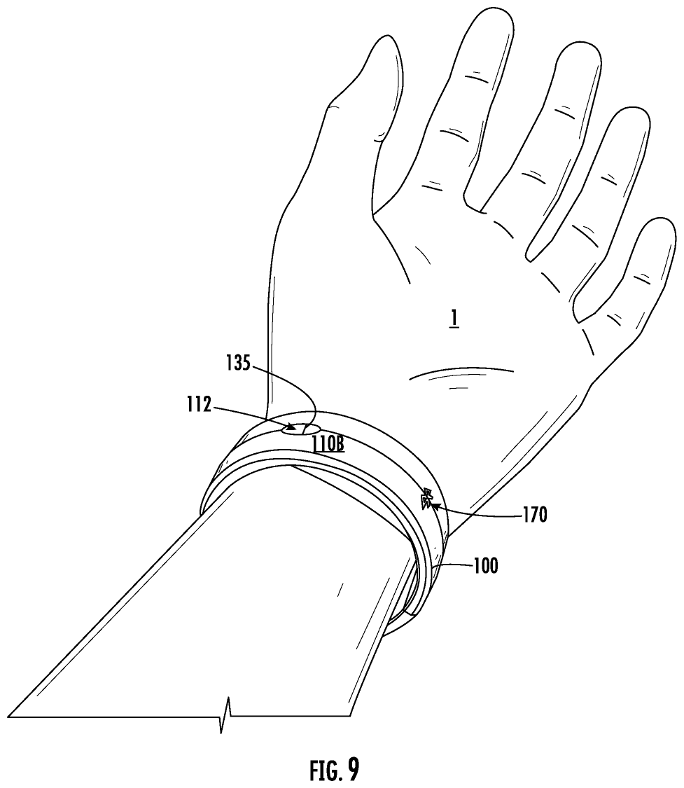

FIGS. 8 and 9 illustrate different views of an example embodiment of the wearable device 100 on the wrist 1 of a user. In FIG. 8, both the indicator 180 (shown by the LEDs under the lens in the shape of a “Z”) and the sample intake port 140 are facing toward the view of the user from the upper surface of the wrist 1. This orientation of the wearable device 100 on the wrist 1 gives the user the visibility to visually see illumination of the indicator 180 to know the level of intoxication of the user. This orientation of the wearable device 100 on the wrist 1 also provides the sample intake port 140 in a position that is facing (e.g., angled and/or inclined) towards the user, as well providing enhanced ergonomics for the user introducing the sample (e.g., by blowing) into the detachable induction tube. In some embodiments, the wearable device 100 is configured to be used by the user (e.g., by blowing into the induction tube) while the wearable device 100 remains positioned on, around, and/or about the user's wrist 1, hand, finger, etc. FIG. 9 shows the lower of an example embodiment of the wearable device 100 on the user's wrist 1. As shown in FIG. 9, the integrated holster 112 is positioned on the lower surface of the user's wrist 1 (e.g., the same side of the wrist on which is the user's palm).

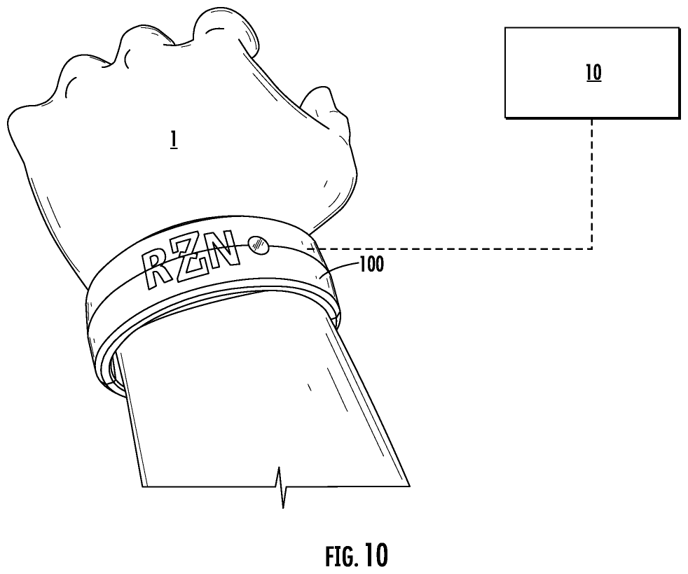

Referring to FIG. 10, a system is shown, the system comprising one or more wearable devices 100 according to the example embodiment discussed elsewhere herein in communication with a computing device 10, such as, for example and without limitation, a mobile smartphone. In some embodiments, as described elsewhere herein, the wearable device 100 communicates analysis results (e.g., as obtained using the analysis compartment contained within the first housing 120) to the mobile smartphone via Bluetooth®, WiFi®, or any other suitable wireless communication protocol. In some embodiments, the computing device 10 has stored in a memory thereof a software application (e.g., a mobile software application) as described above, which software application is executed by one or more processors of the computing device 10 and receives and/or displays information to a user via a display (e.g., a touchscreen). In some embodiments, the mobile software application comprises a signature feature such that consent for sexual acts with the user can be given if the wearable device 100 has performed a chemical composition analysis on a sample obtained from the user within a predetermined period of time and has determined that the level of intoxication of the user is at or below (e.g., no greater than) a predefined level of intoxication, which can be a pre-stored, or default value, or a user-input value. Additionally, in some embodiments, the mobile software application includes a photographic and/or video feature, wherein sexual partners could indicate their consent either on video or, otherwise, photographically. Such a feature would be advantageous in providing enhanced consent of the participants in the sexual act that can be readily verified, thereby providing enhanced safe sex practices.

In some embodiments, the mobile software application is configured to receive data from the user to help the mobile software application determine the intoxication level of the user. Such data can include, for example and without limitation, weight, height, gender, recent food intake, biological/genetic risk, and recent hydration. When the mobile software application has received the results of the sample analysis of the user from the wearable device 100, the mobile software application can use the analysis, along with the user inputs, to determine what is the level of intoxication of the user and instruct the wearable device 100 to illuminate the indicator 180 (e.g., particular LEDs of the indicator 180) on the wearable device 100 to indicate what is the level of intoxication and/or the type (e.g., source) of intoxication affecting the user.

In some embodiments, the mobile software application is configured to receive a user-input value for a desired limit to the level of intoxication of the user. According to this example embodiment, one or more LEDs of the indicator 180 can be used to indicate intelligent suggestions. For example and without limitation, the LED(s) of the indicator 180 can indicate not only the level of intoxication of the user, but also suggest what the user should do based on their level of intoxication. For example, if the user's intoxication level is determined to be greater than the legal limit for intoxication to operate a vehicle, the indicator 180 can illuminate one or more LEDs that would correspond to a suggestion for the user to seek alternative transportation methods (e.g., to hail a taxi, call a friend, etc.).

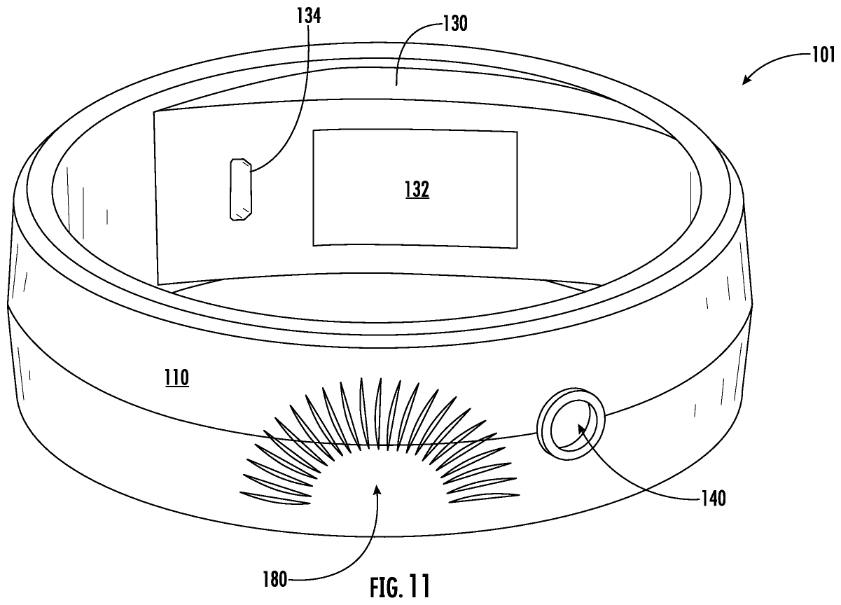

FIG. 11 shows a modified example embodiment of the wearable device shown in FIGS. 1-10. Thus, in FIG. 11, the wearable device is generally designated 101 and is configured for detecting intoxication of the wearer. In FIG. 11, the wearable device 101 comprises a band 110 that is formed as a continuous and uninterrupted annular structure, or ring. Formed in a surface of, and through a thickness of (e.g., partially or entirely), the band 110 is a sample intake port 140, which has an inlet surface that extends beyond (e.g., radially) an outer surface of the band 110. The band 110 has a housing 120 in which is contained a rechargeable battery 122 and a power/data interface port 134 (e.g., in the form of a micro-USB port, or any other suitable type of data/power communications port). The indicator 180 is generally in the shape of a plurality of slits that are circumferentially arranged in a semi-circular pattern. Thus, since the example embodiment shown in FIG. 11 has 23 individual slits (e.g., each slit being illuminated independently of any other slit by one or more LEDs), the illumination of each individual slit can correspond to progressively greater levels of intoxication as determined by the wearable device 100. Fewer or more slits of the indicator 180 can be included from the indicator 180 shown. In some embodiments, some portion of the slots may be dedicated to a presentation of a level of intoxication due to ingestion of alcohol, while others of the slots may be dedicated to presentation of information regarding the detection of particular other intoxicants (e.g., Rohypnol, Benzodiazepine, Ketamine, THC, etc.) during sample analysis. The features of the wearable device 101 may be combined without limitation with any other features of the wearable devices disclosed herein, including communication with a mobile software application operating on a mobile electronic device (e.g., smartphone).

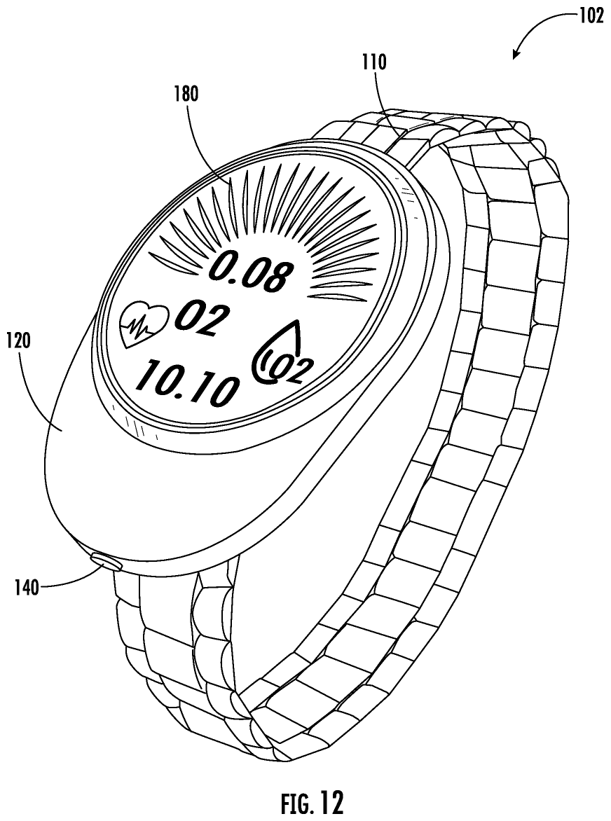

FIG. 12 shows aspects of another example embodiment of a wearable device, generally designated 102, used to detect intoxication of the wearer. According to this example embodiment, the wearable device 100 comprises a band 110 which is connected to a housing 120. The band 110 can have an adjustable length (e.g., due to the inclusion of elastics and/or the removal of links thereof) and is attached at the opposing ends thereof to the housing 120. The housing 120 has a sample intake port 140, which is substantially similar to other sample intake ports 140 of other example embodiments disclosed herein, and which is in communication with an analysis compartment contained therein for performing chemical composition analysis of a sample (e.g., exhaled breath of the wearer) to determine a level of intoxication of the wearer. The sample intake port 140 can be exhaled directly into by the user or with the aid of an induction tube (see 135, FIGS. 6 and 7). The housing 120 further comprises a rechargeable battery and a power/data interface port. In order to allow enhanced physical access to the sample intake port 140, at least one end of the band 110 is connected away from the edge of the housing 120 in which the sample intake port 140 is attached. The housing 120 further includes an indicator 180, in the form of a display (e.g., made using any of an LCD, OLED, micro-LED, or other suitable display technology). The indicator 180 is configured to show, numerically and/or graphically, the level of intoxication of the wearer as determined based on the results of analysis of the sample, as well as any other suitable biometric information of the wearer, including, for example pulse, oxygen saturation, blood glucose, etc. The features of the wearable device 102 may be combined without limitation with any other features of the wearable devices disclosed herein, including communication with a mobile software application operating on a mobile electronic device (e.g., smartphone).

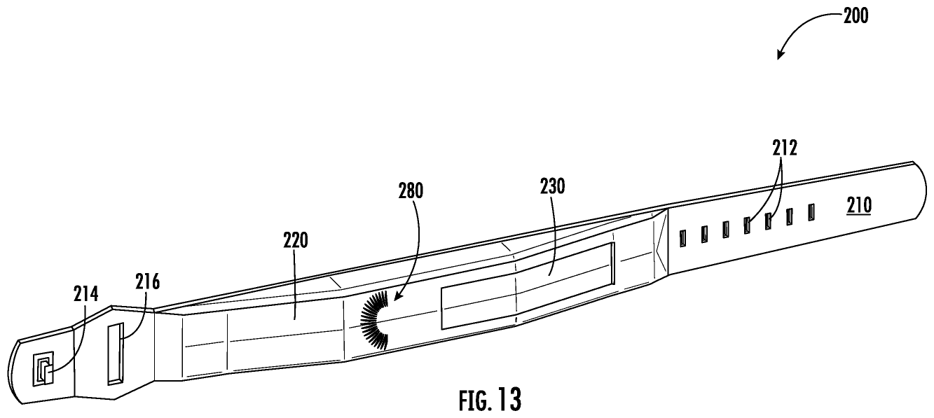

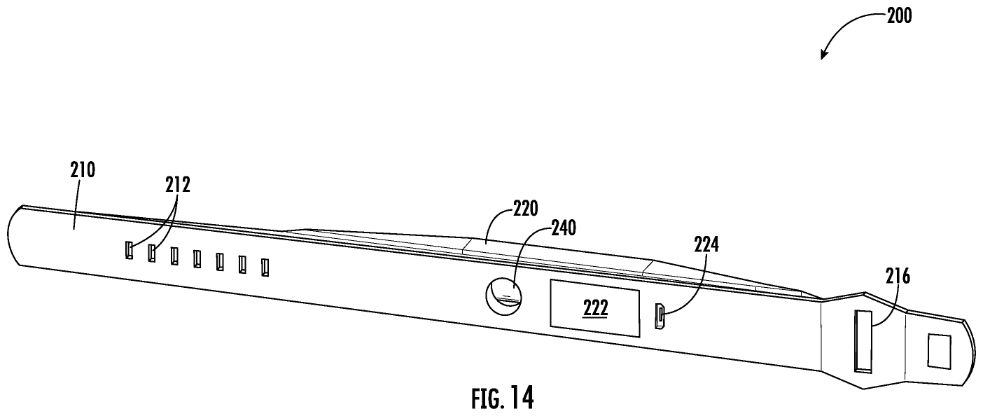

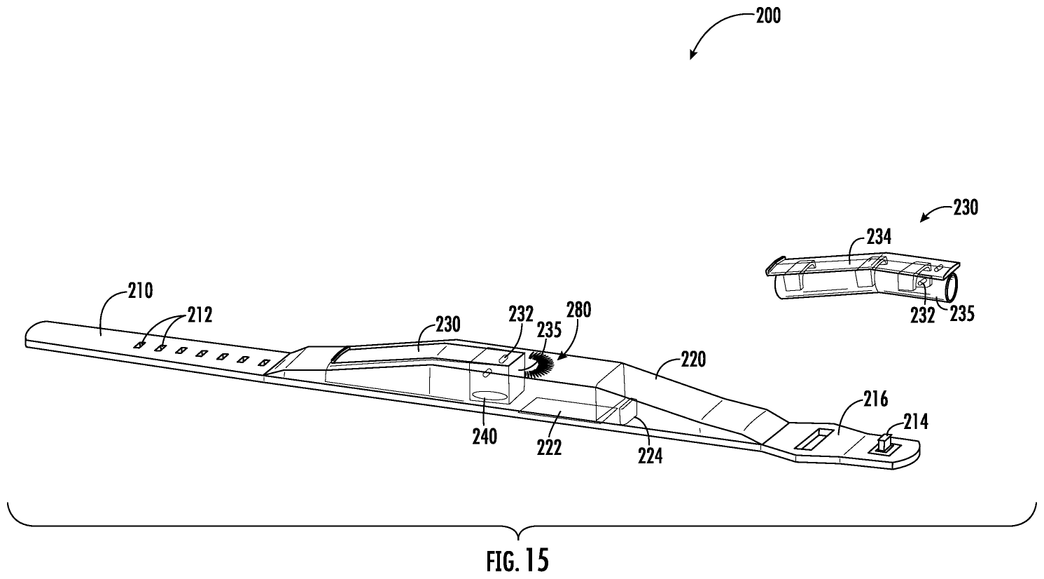

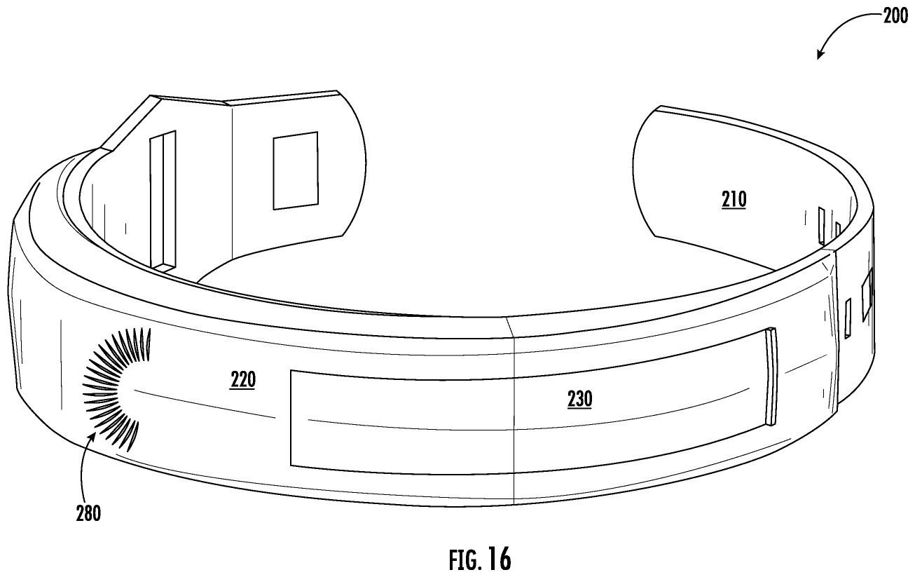

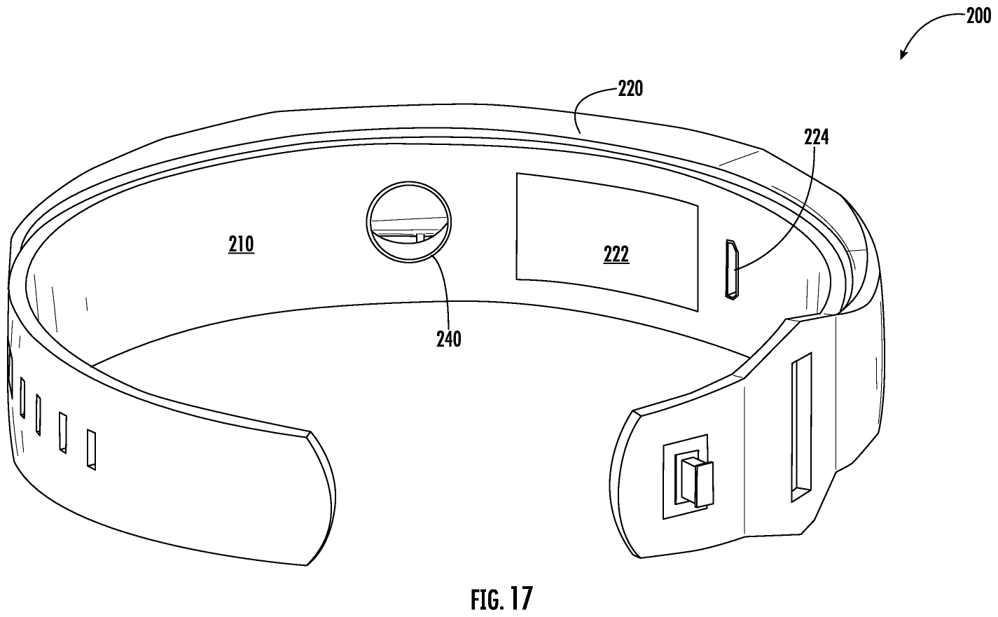

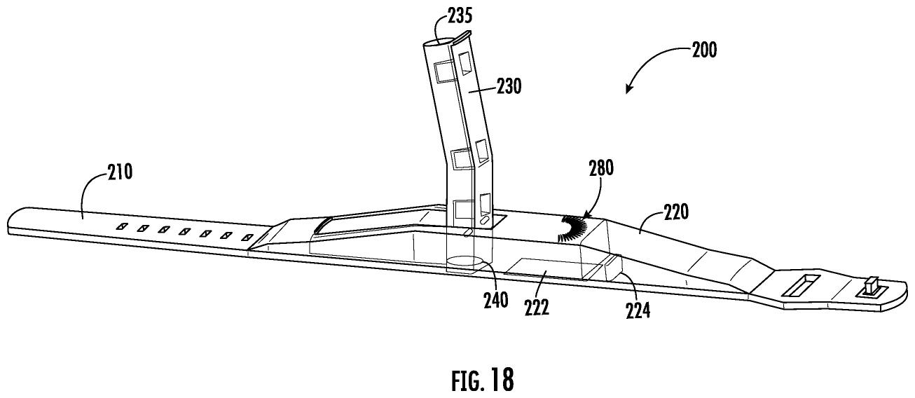

In another example embodiment, shown in FIGS. 13-18, a wearable device, generally designated 200, used to detect intoxication of the wearer is provided. The wearable device 200 comprises a strap 210 with a housing 220 rigidly attached thereto. The strap 210 has a plurality of slots 212 and a fastener 214 provided on opposite sides of the housing 220. The housing 220 (e.g., partially or entirely) and the strap 210 comprise a pliable material that allows for the strap 210 and the housing 220 to be wrapped around (e.g., conform to) the wrist of a wearer when the wearable device 200 is being worn. The fastener 214 is configured for engagement within any of the slots 212 of the strap 210 to allow for the wearable device 200 to be worn by users having any suitable wrist circumference. An excess strap slot 216 is formed through the strap 210, adjacent to the fastener 214, such that any excess strap can be tucked under the strap 210 when the wearable device 200 is worn by the user. The housing 220 has a rechargeable battery 222 and a power/data interface port 224. The power/data interface port 224 is accessible through the strap 210. The wearable device 200 comprises an induction tube assembly 230, which comprises an induction tube 235. The induction tube assembly 230 is not detachable from the housing 220, but is instead pivotably attached (e.g., via pins 232) thereto. The induction tube assembly 230 comprises a cover 234, which has a same outer contour as the portion of the housing 220 immediately surrounding the induction tube assembly 230. The cover 234 is rigidly attached to the pins 232 and the induction tube 235, such that the induction tube 235 and the cover 234 rotate in unison as the induction tube assembly 230 pivotably rotates about the pins 232. The strap 210 and/or housing 220 have formed therein a sample intake port 240, with which the induction tube 235 is selectively engageable based on an angular position of the induction tube assembly 230. As shown in FIG. 13, the induction tube assembly 230 is in a stowed position, such that the induction tube 235 attached thereto is not engaged with (e.g., is not in fluid communication with) the sample intake port 240. FIG. 18 shows the induction tube assembly 230 in a deployed position, in which the induction tube 235 attached thereto is engaged with (e.g., in fluid communication with) the sample intake port 240. The wearable device 200 further comprises, on the housing 220, an indicator 280. The indicator 280 can be the same as or different from any of the other indicators (e.g., 180) illustrated and described elsewhere herein. In the example embodiment, the indicator 280 comprises a plurality of semi-circularly arranged LEDs that are illuminated in a prescribed pattern to indicate the level of intoxication detected by the wearable device 200 and/or the presence of one or more specific intoxicants having been detected in the sample by the wearable device 200. The features of the wearable device 200 may be combined without limitation with any other features of the wearable devices disclosed herein, including communication with a mobile software application operating on a mobile electronic device (e.g., smartphone).

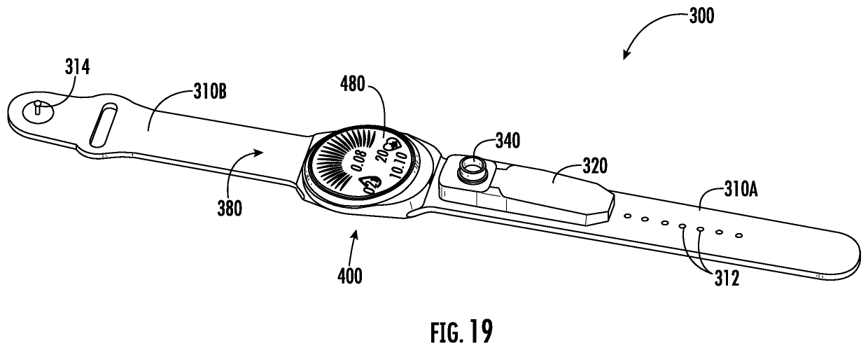

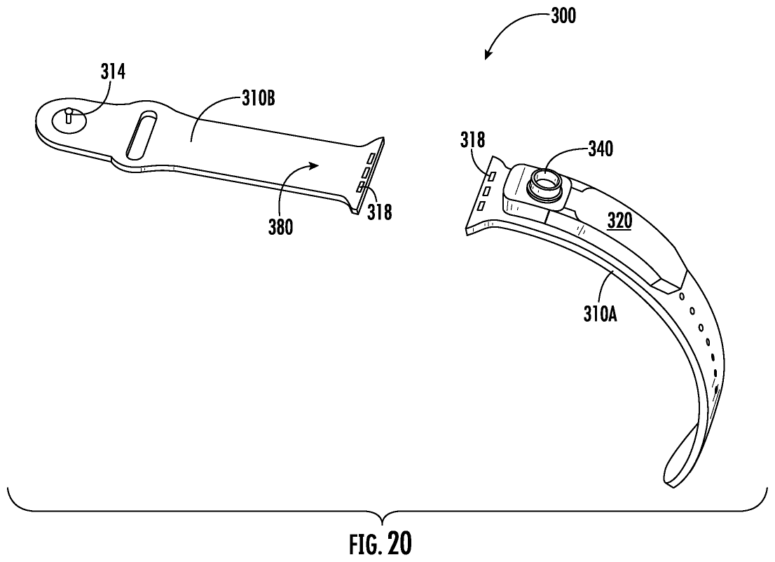

FIGS. 19 and 20 show an example embodiment of a wearable device, generally designated 200, used to detect intoxication of the wearer, as integrated into a smartwatch, generally designated 400. The wearable device 300 comprises first and second straps 310A, 310B, which are attached on opposing sides of a housing of a smartwatch 400. The first and second straps 310A, 310B comprise contacts 318 that can mechanically interlock with the housing of the smartwatch 400 and also serve as electrical contacts for power and/or data communication with the smartwatch 400 (e.g., for displaying results of the analysis of the sample on the indicator 480 (e.g., the display) of the smartwatch 400. The first strap 310A comprises a housing 320 rigidly attached thereto. The first strap 310A has a plurality of slots 312 and the second strap 310B has a fastener 314, such that the slots 312 and the fastener 314 are positioned on opposite sides of the housing 320. The housing 320 (e.g., partially or entirely) and the first and second straps 310A, 310B comprise a pliable material that allows for the first and second straps 310A, 310B and the housing 320 to be wrapped around (e.g., conform to) the wrist of a wearer when the wearable device 300 attached to the smartwatch 400 for wearing about the wrist of a wearer. The fastener 314 is configured for engagement within any of the slots 312 to allow for the wearable device 300 to be worn by users having any suitable wrist circumference. In some embodiments, the housing 320 has a rechargeable battery and a power/data interface port. In the example embodiment shown, however, the housing 320 has neither a power source nor a power/data interface port associated therewith, the wearable device 300 receiving power and data communication via the connection (e.g., using contacts 318) with the smartwatch 400. The housing 320 has formed therein a sample intake port 340, in which a sample from the user can be provided directly and/or via use of an induction tube (e.g., 135, FIGS. 6 and 7). The wearable device 300 is configured to transmit results of the analysis of the sample obtained from the user to the indicator 480 (e.g., the display) of the smartwatch 400 for displaying the results of the analysis to the user. The indicator 480 is advantageously a graphical display (e.g., made using any of an LCD, OLED, micro-LED, or other suitable display technology) and can be, in some embodiments, a touchscreen. The indicator 480 is configured to show, numerically and/or graphically, the level of intoxication of the wearer as determined based on the results of analysis of the sample, as well as any other suitable biometric information of the wearer, including, for example pulse, oxygen saturation, blood glucose, etc. The features of the wearable device 300 may be combined without limitation with any other features of the wearable devices disclosed herein, including communication with a mobile software application operating on a mobile electronic device (e.g., smartphone).

In some embodiments, the example embodiments of the wearable devices disclosed herein include one or more (e.g., a plurality of) LEDs for use as all or a portion of the indicator (e.g., display).

In some embodiments, the example embodiments of the wearable devices disclosed herein comprise a digital clock screen.

In some embodiments, the example embodiments of the wearable devices disclosed herein comprise an indicator in the form of a display in the form of a touchscreen. In some such embodiments, the display is a full smartwatch touchscreen.

In some embodiments, the example embodiments of the wearable devices disclosed herein may be integrated into, for example, an Apple Watch®, Samsung Galaxy® Watch, and/or Fitbit® accessory (e.g., activity tracker and/or smartwatch).

In some embodiments, the example embodiments of the wearable devices disclosed herein comprise straps that have a semi-circular elastic design.

In some embodiments, the example embodiments of the wearable devices disclosed herein comprise straps that use standard band designs.

In some embodiments, the example embodiments of the wearable devices disclosed herein use a smart signal LED display for the indicator (e.g., 180, 280), which shows colors that correspond to the desired limit(s) of intoxication, as specified by the user or using default threshold values. The smart signal LED display can be in the form of a corporate logo, presented on a display, as a single dot (e.g., lens), or any other form.

In some embodiments, the example embodiments of the wearable devices disclosed herein can be modified, as will be understood by persons having ordinary skill in the art, for wearing on, around, and/or about finger(s) (e.g., in the manner of a ring), wrist, or neck (e.g., in the manner of a necklace) of the user.

In some embodiments, the sensor(s) and/or circuit(s) configured for detecting the presence and/or concentration of an intoxicant, for example, alcohol, can be in the form of an alcohol detection sensor system that is configured to detect the presence and/or concentration of alcohol in the blood of the user without requiring the user to provide an exhaled sample for analysis. In some such example embodiments, the alcohol detection sensor system is configured to detect the presence of alcohol via (e.g., through, such as by direct contact with) the skin of the user and, optionally, the concentration of alcohol detected. Thus, in some such example embodiments, the alcohol detection sensor system comprises one or more sensors that are provided adjacent to, or on the surface of (e.g., to have a surface of such one or more sensors that is coplanar to and/or protrudes beyond a surface of), the inner surface (e.g., the surface against the skin of the user when such wearable device is worn about, for example, the wrist of the user) of the band or strap(s). In some such example embodiments, the one or more sensors have at least one surface thereof exposed (e.g., uncovered by the band or strap(s)) for detection of alcohol through the skin of the user. As used herein, the term “sensor” includes any device, component, and/or structure (e.g., including electrical wire and/or circuitry components) by which the presence and/or concentration of alcohol in the blood of the user can be detected through skin (e.g., human skin) of the user by being positioned adjacent thereto and/or in direct contact therewith.

In some embodiments, the example embodiments of the wearable devices disclosed herein comprise a spring mechanism by which the induction tube (e.g., 135, 235) can be maintained in a flush position with respect to the surface of the band of the wearable device, or to protrude therefrom in a way that better aligns with the analysis unit of the wearable device.

In some embodiments, the mobile software application is configured to transmit, in realtime and/or periodically, the results of the analysis of the user to one or more persons specified by the user. Such one or more persons can be designated individuals that the user trusts to monitor the level of intoxication of the user.

Unless defined otherwise, all technical and scientific terms used herein have the same meaning as commonly understood to one having ordinary skill in the art to which the presently disclosed subject matter belongs. Although, any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in this application, including the claims. Thus, for example, reference to “a vial” can include a plurality of such vials, and so forth.

Unless otherwise indicated, all numbers expressing quantities of length, diameter, width, and so forth used in the specification and claims are to be understood as being modified in all instances by the terms “about” or “approximately”. Accordingly, unless indicated to the contrary, the numerical parameters set forth in this specification and attached claims are approximations that can vary depending upon the desired properties sought to be obtained by the presently disclosed subject matter.

As used herein, the terms “about” and “approximately,” when referring to a value or to a length, width, diameter, temperature, time, volume, concentration, percentage, etc., is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate for the disclosed apparatuses and devices.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and sub-combinations of A, B, C, and D.

The present subject matter can be embodied in other forms without departure from the spirit and essential characteristics thereof. The embodiments described therefore are to be considered in all respects as illustrative and not restrictive. Although the present subject matter has been described in terms of certain specific embodiments, other embodiments that are apparent to those of ordinary skill in the art are also within the scope of the present subject matter.

1. A wearable device for measuring intoxication of a user, the wearable device comprising:
a band comprising a sample intake port;
one or more processors or other circuitry configured to analyze a sample from the user, the sample being inducted into the sample intake port, and to determine a level of intoxication of the user based on an analysis of the sample; and
an indicator configured to visually display the level of intoxication of the user.

2. The wearable device of claim 1, comprising a detachable induction tube configured to extend outward from the band when inserted into the sample intake port.

3. The wearable device of claim 2, wherein the band further comprises an integrated holster configured for securely holding the detachable induction tube in a position such at least a portion of the detachable induction tube is hidden within the band.

4. The wearable device of claim 1, wherein the band comprises two semi-circular band portions that are interconnected at opposing ends thereof with a resilient member and held together with magnets.

5. The wearable device of claim 1, wherein the band is configured to be worn on a wrist, hand, or one or more finger of the user.

6. The wearable device of claim 1, wherein the indicator comprises one or more light emitting diodes (LEDs) on an exterior surface of the band.

7. The wearable device of claim 6, wherein the indication of the level of intoxication of the user is based on user input settings.

8. The wearable device of claim 6, wherein the one or more LEDs comprises a plurality of LEDs, each of which is a different color to indicate various levels of intoxication.

9. The wearable device of claim 8, wherein:
the level of intoxication represented by each of the plurality of LEDs can be set by the user using a software application in communication with the wearable device via Bluetooth, WiFi, and/or a mobile communications data network; and
the wearable device is configured to transmit results of analysis of the sample to the software application for determining the level of intoxication of the user based on the analysis.

10. The wearable device of claim 9, wherein the mobile application is stored in a memory of, and executed by one or more processors of, a mobile phone, tablet, personal digital assistant, or laptop.

11. The wearable device of claim 9, wherein the mobile application comprises a signature feature such that sexual consent can be indicated via the signature feature only when the level of intoxication of the user is below a predetermined threshold.

12. The wearable device of claim 1, wherein the level of intoxication is determined based on presence and/or concentrations of alcohol, tetrahydrocannabinol (THC), Rohypnol, Benzodiazepine, Ketamine, and/or opioids in the sample.

13. The wearable device of claim 1, wherein the indicator comprises a display screen.

14. The wearable device of claim 13, wherein the display screen is a touchscreen.

15. The wearable device of claim 1, wherein:
the band is configured for attachment to a smartwatch; and
the indicator is a display of the smartwatch.

16. The wearable device of claim 1, wherein the band comprises first and second housings attached rigidly to an interior surface of the band, the first housing comprising a power source and the second housing comprising an analysis compartment in fluidic communication with the sample intake port for receiving the sample within the analysis compartment.

17. The wearable device of claim 16, wherein the first and second housings have a shape such that a distance between midpoints of a radially inward facing surface of each of the first and second housings less than a diameter of the band.

18. The wearable device of claim 17, wherein the shape of the first and second housings resist rotation of the wearable device about a wrist of the user when the wearable device is positioned on, about, and/or around the wrist of the user.

19. A system for determining a level of intoxication of a user, the system comprising:
the wearable device according to claim 1; and
a computing device operating a software application;
wherein the wearable device is in communication with the computing device and the software application and is configured for determining the level of intoxication of the user.

20. The system of claim 19, wherein the computing device is a mobile device and the software application is a mobile software application.
